(19) United States
(12) Patent Application Publication
(10) Pub. No.: US 2021/0161950 A1
(43) Pub. Date: Jun. 3, 2021

(54) Veterinary Supplement for Effecting Bone and Cartilage

(71) Applicant: Lewis Kilman Clarke, Friendswood, TX (US)

(72) Inventor: Lewis Kilman Clarke, Friendswood, TX (US)

(21) Appl. No.: 16/954,063

(22) PCT Filed: Dec. 15, 2018
(86) PCT No.: PCT/US18/65890

§ 371 Date: Jun. 15, 2020

Related U.S. Application Data
(60) Provisional application No. 62/599,484, filed on Dec. 15, 2017.

(51) Int. Cl. A61K 33/00; A61K 31/7008; A61K 31/737; A61K 31/07; A61K 31/593; A61K 31/122

(52) U.S. Cl. CPC A61K 33/00; A61K 31/7008; A61K 31/122; A61K 31/07; A61K 31/593; A61K 31/737

(57) ABSTRACT

The present invention is a formulation containing the essential and necessary ingredients, in combination, exhibiting chondroprotective and chondro-regenerative properties for bone, cartilage, and joint maintenance and repair. The formulation further promotes overall bone, cartilage and joint health in an effort to remedy the effects of natural degeneration, immunologically weakened structures as well as osseous damage due to repetitive physical stress, illness or traumatic injury.

### BACKGROUND OF THE INVENTION

### Field of the Invention

The present invention generally relates to a formulation used as a veterinary dietary supplement and nutritional adjuvant to support the growth, maintenance and repair of bone and cartilage. More specifically, the present invention is drawn to formulations that comprise novel combinations of ingredients, in the form of micronutrients, having a complex biochemical interdependence with one another which are used for maintaining bone density (in avoidance of fractures and osteoporosis), supporting lubricity in cartilage, and maintenance of an animal's healthy coat, skin, teeth, bones and eyes or a combination thereof. Efficacy of the formulation is directly related to individual micronutrients alone and in symbiotic combination.

### Background

A vast number of domesticated animals, including commensals bred for human companionship (e.g. domesticated animals including dogs and cats), prey animals for human consumption (e.g. cows, pigs, sheep and goats), and draft animals such as horses, mules and camels, at some point develop some manner of bone, joint and/or cartilage deterioration over the course of their lives. Routinely, the vast majority of these conditions occur in direct proportion to animal age and corresponding wear over time. Reasons for the degradation and degeneration of bone and joints vary and result from multi-faceted etiologies that range from (1) declines in bone and joint integrity due to age and prolonged mechanical stress, (2) protracted immune disorders, (3) more immediate causes including traumatic injuries due to sprains, strains, dislocations and fractures and (4) as a result of acute and protracted infection.

Regardless of etiology, the subsequent manifestations of orthopedic injury, wear and musculoskeletal disorders range from mild discomfort and limited range of motion to transient incapacitation, prolonged immovability to complete immobility. Aside from the resultant pain, the inability to function normally has both immediate and long-lasting effects on a domesticated animal's ability to operate and function normally. And while treatment options vary widely depending on origination and severity, the correction of the underlying malady falls abjectly in one of 3 categories: repair, maintenance and prevention. Fortunately, remediation, upkeep and prevention can all benefit from one source—dietary supplementation with a specific combination of micronutrients that have been shown to not only remediate and repair orthopedic sequelae but also to promote and support bone growth. Moreover, it is not merely the inclusion of each micronutrient, singly, that accounts for their overall effectiveness but a symbiotic, co-dependent relationship of each to the other that demands the necessity for their concurrent and co-administration.

The present invention, therefore, relies upon the inclusion of several dietary supplement entities to treat the genetic, temporal and traumatic causes of debilitating bone loss or damage. Specifically, these ingredients include, but are not limited to, (1) glucosamine, (2) chondroitin, (3) strontium carbonate, (4) vitamin A, (5) vitamin D3, and (6) vitamin K2 mk7. Equally, while these core six (6) micronutrients are included in the primary formulation as detailed and described, the addition of other osteoblastic, bone supporting and anti-osteoclastic ingredients may as well be included to further augment this original formulation. Each of the aforementioned ingredients is detailed below.

### Glucosamine and Chondroitin

Glucosamine and chondroitin both fall under the category of chondroprotective agents which delay progressive joint space narrowing (a hallmark of arthritis) and improve the structure and function of the mechanical aspects of articular joints through chondrocyte protection. By what mode and to what extent the combination of glucosamine and chondroitin functions to repair and support bone growth and maintenance points to at least one, and more likely several, of the following mechanisms: 1) stimulating chondrocyte synthesis of collagen and proteoglycans, 2) enhancing synoviocyte production of hyaluronan, 3) inhibiting cartilage degradation, and/or 4) preventing fibrin formation in the vasculature.

Both compounds, glucosamine and chondroitin, are classified as structure modifying osteoarthritis drugs (SMOAD) and symptomatic slow acting drugs in osteoarthritis (SYSADOA). Systemically, glycoprotein and glycosaminoglycan (GAG) are derived from glucosamine salts, where glycoproteins play an integral role in connective tissue helping to bind fibers, cells and ground substances (and inorganic substances like calcium in bone) and glycosaminoglycans themselves consist of alternating acetylglucosamine (or N-acetylgalactosamine) together with uronic sugars to form chondroitin sulfate. As well, hyaluronic acid (hyaluronan), itself a member of the class of GAGs, serves as a primary component of synovial tissues, synovial fluid and other soft tissues exhibiting remarkable viscoelastic properties ideal for the lubricating of joints. Chemically, glycosaminoglycans are extremely polar compounds that attract water and function as a lubricant and shock absorber between bones where each is essential to the articular cartilage matrix and to lubricating synovial fluid. Ultimately, glucosamine, and specifically glucosamine production, is the rate limiting step in this process and, as levels of glucosamine decline with age, so exogenous supplementation with glucosamine can overcome impediments in its production thereby counteracting this loss. Subsequently, as would be expected, glucosamine supplementation does, in fact, increase hyaluronic acid in the synovium thus resulting in increased shock absorption between two corresponding bones. In addition, glucosamine also inhibits synthesis of the catabolic and inflammatory matrix metalloproteinases, as well as other inflammatory cytokines, thereby preserving existing proteoglycans. Meta-analysis of papers between 1950 and 2007 show that glucosamine:
1. Reduces pain;
2. Improves mobility of joints;
3. Reduces osteoarthritis progression; and
4. Reduces risk of total joint replacement

Furthermore, glucosamine may, in fact, serve a temporal as well as restorative function. A 2013 study by the Department of Orthopedics and Traumatology at Haseki Training and Research Hospital in Turkey found that glucosamine helped accelerate the healing time it took to heal from bone fractures in rats. Specifically, these researchers found that new bone formation and osteoblast lining were significantly higher in glucosamine-treated rats as compared to those in control groups. After 4 weeks of taking 230 milligrams of glucosamine sulfate daily, the rats' connective tissue surrounding bones was more cellular and vascular and the newly formed bones that were previously fractured were stronger compared to controls. Manifestly, glucosamine and chondroitin are structural components of cartilage, function as SMOAD and SYSADOA drugs, and, when used along with Strontium (evidenced below), can effectively rebuild bone and depleted joint's cartilage structures. Further, chondroitin is a major component of cartilage that helps it retain water, increasing its cushioning and lubricating qualities.

In addition to hyaluronan, it has long been observed that chondroitin sulfate is yet another naturally occurring glycosaminoglycan (GAGs) existing in the connective tissue (e.g. tendons, ligaments, cartilage and bone) within and between bones and, like hyaluronic acid, chondroitin decreases pain and inflammation, but in addition, it too slows the development of osteoarthritis.

By far the most prevalent GAG, chondroitin inhibits destruction of cartilage and stimulates new cartilage formation in a dose-dependent manner to increase cell proliferation as well as supplanting an essential building block in cartilage. Chondroitin, like glucosamine, also stimulates the synthesis of proteoglycans and hyaluronic acid by inhibiting hyaluronidase (and other catabolic enzyme activity) in synovial fluid in addition to its anti-inflammatory actions and overall reduction in catabolic activity of chondrocytes or, alternatively, chondroitin's cartilage fostering maintenance functions.

### Strontium Carbonate

The stable form of strontium, an alkaline earth metal similar in structure to calcium, works by accumulating in bone (specifically where active bone remodeling occurs), directly suppressing the activity of the ‘osteoclast’ (i.e. the cells that dissolve and promote deossification). This aids in bone growth and increases bone density thereby decreasing the rates and incidences of bone fracture. The suppression by strontium of bone resorption by inhibition of osteoclastic activity occurs by 3 mechanisms: decreased differentiation of stem cells into osteoclasts, decreased activity of existing osteoclasts, and decreased survival (or increased apoptosis) of osteoclasts. Furthermore, strontium stimulates both differentiation of stem cells into osteoblasts and the replication of these osteoblasts as well as their survival via the CaSR or the calcium sensing receptor. Strontium, therefore ‘uncouples’ bone resorption from bone formation. Strontium does this while maintaining bone and without adversely altering bone matrix mineralization. Strontium, then, substitutes for the calcium in the matrix, increasing its density without compromising its microarchitecture and fracture-resistant flexibility.

In addition, researchers have found that strontium improves cartilage metabolism in osteoarthritis by stimulating production of proteoglycan (structural components of cartilage such as glucosamine and chondroitin above) and actually alters the composition of the proteoglycans, demonstrating the ability of strontium to stimulate the chondrocytes to produce a matrix of different macromolecular composition. As well, in studies strontium has been shown to be a powerful stimulus of human cartilage matrix formation and, it should be noted, that this occurs by an ionic effect without concurrent induction of unwanted cartilage resorption or chondroresorption.

Liu, et.al. in 2016, studying the degenerated cartilage of the mandibular joint, found that treatment with oral strontium not only reversed the subchondral bone loss, but also the mandibular condylar cartilage degradation as evidenced by increased cartilage thickness and cell density. Liu also noted increased expression of cartilage matrix molecules such as Collagen II and aggrecan and decreased expression of cartilage catabolic factors and pro-inflammatory cytokines.

Succinctly, in the presence of strontium, bone and cartilage growth, maintenance and healing processes are directly facilitated through the stimulation of bone generating cells, the osteoblasts, and inhibition of bone dissolving cells, the osteoclast, which results in improved bone density in osteoporosis and enhanced fracture healing. From related studies it appears there exists a strong indication that these anabolic effects of strontium are due to synergistic interaction with other micronutrients such as glucosamine and chondroitin as well as vitamins A and D and K2 (Genuis and Bouchard, 2011).

### Vitamin A

Vitamin A, is a group of unsaturated nutritional organic compounds that includes retinol, retinal, retinoic acid, and several provitamin A carotenoids, which are responsible for making new tissue, promoting muscles, nerves, skin and mucous membrane maintenance and growth, supporting proper functioning of the immune system and aiding in healthy vision. Equally, Vitamin A plays an essential role in the development of osteoblasts, the bone-building cells that generate new bone. Deficiencies in vitamin A can occur as a primary or secondary deficiency (primary deficiency occurring through inadequate intake of vitamin A and secondary deficiency occurring through malabsorption). Absence of proper levels of vitamin A result in poor bone growth by limiting calcium absorption and metabolism, which, in turn, weaken bone and allow for an increased risk for untoward bone related events.

However, without vitamin D, Vitamin A supplementation can also cause bone resorption which is undesirable in fracture or dysplasia repair. This is an important observation that only in the presence of vitamin D does Vitamin A promote osteogenesis or bone formation. The explanation for this is found in a complex interaction between Vitamin D, Vitamin A, Vitamin K2 and the thyroid. Chemically, Vitamin A can be observed to wrap around vitamin D and vitamin K2, together with a thyroid molecule, a process known as heterodimerization, and this heterodimer enters the osteoblast, triggering the DNA of that cell to start generating bone. Absent these described events, precipitated by low vitamin A levels or vitamin A without supplementation of vitamin D, osteoporosis and increased risk of fracture, especially in older mammals is the natural result. For the osteoblast to generate bone, vitamin A, vitamin D, vitamin K2 must be available simultaneously thus demonstrating the interdependence of these supplements and the absolute necessity of their co-inclusion and co-administration.

Moreover, it is interesting to note that osteoblasts, the bone “making” cell, and the adipocyte, the fat cell, are derived from the same stem cells. Vitamin A, vitamin K2 and strontium all promote the differentiation of these stem cells in favor of the osteoblast rather than the adipocyte thereby moving the organism toward a requisite anabolic repair of bone and away from fat storage. This is again another example of the required symbiosis of these supplements apart and above their individual inclusion in the present formulation.

### Vitamin K2 mk7

Vitamin K2, or menaquinone, is in fact a group of 9 related homologues involved in the synthesis of proteins that regulate bone metabolism. Vitamin K2 has dual actions on bone. First, in conjunction with vitamin D and vitamin A, it directly promotes bone formation by stimulating the differentiation of stem cells into osteoblasts. Vitamin K2 upregulates, in these new cells, the expression of the bone marker genes, and functions as an essential co-factor of several bone proteins involved in calcium uptake and bone matrix mineralization. Specifically, the MK-7 form of vitamin K2, expressly included in the formulation of the present invention, upregulates the SXR target gene, CYP3A4, in the osteoblasts which induces the production of osteocalcin. Osteocalcin is a non-collagenous protein hormone secreted solely by osteoblasts and which plays a pivotal role in the body's metabolic regulation of pro-osteoblastic (bone-building) capabilities, bone mineralization and calcium ion homeostasis. While transcription and translation of the osteocalcin gene CYP3A4 are regulated by vitamin D3, its ability to bind calcium ions and organize the extracellular matrix and even modulate the size and shape of the hydroxyapatite crystals is dependent on the vitamin K. Most importantly, it should be noted that both vitamin D and vitamin K must be present for this gene to be activated and for bone to be generated.

The second action of vitamin K2 on bone is inhibition of bone resorption. Not only is vitamin K2 known to increase bone mineral density (BMD) but it also has been shown to reduce bone loss through, among other mechanisms, the inhibition of the osteoclast by upregulating the CYP3A4 gene resulting in the synthesis of osteoprotegerin which downregulates expression of NF-kB (decreasing osteoclast formation). Another mechanism by which vitamin K2 inhibits bone resorption is through its anti-inflammatory action on pro-inflammatory prostaglandin E2 and cytokines such as matrix metalloproteinases and IL1alpha. Vitamin K2, therefore has a preventive role for bone demineralization with aging and a facilitatory role in fracture healing.

Additionally, vitamin K2 exerts a powerful influence on not only bone building (osteogenesis), but also cartilage building (chondrogenesis) through its synergistic role with vitamins D and A, calcium, magnesium and zinc. In this regard, one of the 14 known vitamin K dependent proteins is matrix Gla protein which is synthesized by chondrocytes and vascular smooth muscle cells. It has been shown in both human and animal studies to inhibit the calcification of arterial media and cartilage thereby helping to maintain these soft tissues in their normal functional states. Vitamin K2 also stimulates production of both Type 1 collagen for tendons, ligaments and endomysium of myofibrils and Type 2 collagen for articular and hyaline cartilage.

### Vitamin D3

Vitamin D, a group of fat-soluble secosteroids including cholecalciferol and ergocalciferol, stimulates both the absorption of calcium (as well as magnesium and phosphate) from the intestines and the conservation of calcium in the kidney thereby helping the body to retain it. Because of its interplay with calcium, Vitamin D is known to be extremely important in bone formation (growth and remodeling) and vital to efficient muscle control. Low levels of Vitamin D, then, will naturally result in bone demineralization up to and including human disorders osteomalacia in adults and Rickets in children.

This physiology and biochemistry of vitamin D is well known and extensively reviewed elsewhere in numerous texts. Lesser known and relatively recent findings are vitamin D's fascinating interaction with Vitamin A, K2 and thyroid. This is not only critical in the development and repair of bone, but appears to be equally important in cell growth, stem cell regenerative processes, proper neuromuscular and immune system functioning, retarding the inflammation process and cascade and is integral in almost all tissue health and maintenance functions including within the lining of the blood vessels, the brain and nerves.

The scientific and clinical literature in the past 25 years has demonstrated beyond any question that vitamin D is essential to all the above aspects of biochemical function and cellular repair. The intricacies of the vitamin D receptor and the response elements of the receptor and their interaction with other micronutrients in these processes continues to be an ever-expanding universe. It is now becoming clear that the maximal benefits of vitamin D are obtained when these synergistic biochemicals are also present simultaneously and proportionally. Accordingly, the actions of each of the components in this invention are noted in this cursory review to be, in some fashion, all dependent on the presence and actions of vitamin D. Each of these together in comodulation and coactivation induce as yet innumerable cascades of effects in countless intricately interwoven systems of endocrine, cellular physiology and metabolism and micro and macromolecular structures and chemistry. While each individual component has its role, it would be difficult to not place vitamin D in the center, the imperator, as it were, of these many physiologies. And it is certain that many more pivotal roles and interactions will be found as vitamin D research proceeds. For this invention, it is clear that vitamin D must be a component part.

Therefore, while there has been research published in peer-reviewed literature on each of the aforementioned compounds individually, it is the goal of the present formulation to use each component in combination with each other listed component to realize the full potential of a synergistic relationship where the benefits of the sum exceed those of the individual parts. From this review, it is apparent that each ingredient is modulated, enhanced and augmented by all other ingredients to create a symbiotic relationship that provides unexpected and superior results.

The present invention focuses on a long-felt but unaddressed needs for a new and novel invention that seeks to concentrate on reversing bone and cartilage degradation through a stimulating supplement via several distinct mechanisms: (1) targeting degenerative bone disorders by strengthening bone and increasing bone density through prevention and maintenance, (2) addressing the etiology of osteoporosis by building and rebuilding cartilage, (3) increasing joint lubricity, and (4) aiding in the efficient and expedited repair of bone due to continual stress, autoimmune disorder, infection or traumatic injury.

The present invention combines many known elements into a symbiotic relationship that provides results greater to that of each component alone to, at a minimum, protect bone, cartilage and joints from the effects of osteoporosis, provide relief from the debilitating effects of osteoarthritis, and provide for bone, cartilage and joint regeneration after trauma, illness and injury.

Conventional formulations lack the ability to address the current need in the area of supporting growth, maintenance and repair of mammalian bone, holistically, with a discreetly chosen set of formulation ingredients selected for their individual and combined beneficial effects. Thus, there is a long-felt but significant and un-met need for formulations whose individual components can work in a mutually beneficial, symbiotic and collaborative manner to target immediate and long-term regenerative and restorative pathways toward remunerating bone loss, supporting bone growth and preventing the untimely loss of musculoskeletal function. The present invention is a methodical and well-formulated system of singly and interdependent operating components that satisfies this long standing need in the art.

Further, while the formulation and method of use of the present application is susceptible to various modifications and alternative forms, specific disclosed embodiments have been shown by way of example and are herein described in detail as to the preferred formula. It should be understood, however, that the description herein of specific embodiments is not intended to limit the application to the particular embodiment disclosed, but on the contrary, the intention is to include all modifications, equivalents, and alternatives falling within the spirit and scope of the formulation of the present invention as defined herein by the appended claims.

### SUMMARY OF THE INVENTION

The present invention relates to a dietary supplement consisting of chondro-restorative and chondroprotective compounds that delay bone resorption, potentiate osteoclast break down, and postpone and remediate progressive joint space narrowing (characteristic of arthritis) evidenced through (1) stimulation of chondrocyte synthesis of collagen and proteoglycans, (2) enhanced synoviocyte production of hyaluronan, (3) inhibition of cartilage degradation, and (4) prevention of fibrin formation in the vasculature.

The invention itself provides a considered and calculated combination of chondro-generative and chondroprotective agents, supplied-exogenously to supplant and affect systems endogenously, which are directed toward the growth, maintenance and repair of veterinary mammalian bone, joint and connective tissues. Specifically the present dietary supplement consists of a mixture of remunerative and preventive compounds directed toward remediating the effects of osteoarthritis, osteoporosis, inflammatory joint disease, ankylosing spondylitis, and various autoimmune disorders (e.g. rheumatoid arthritis, lupus, and Sjogrens syndrome), promoting preventative states of negative bone loss and/or enhanced bone deposition, and aiding in the promotion of the body's natural state of healing and rehabilitation of bone from mechanical wear, degeneration, strains, fractures, illness and bone breakage.

It is to be understood that the dietary supplement of the present invention can also be prepared and administered with any pharmaceutically acceptable carrier or carriers. Moreover, a dietary supplement of the present invention can also be prepared in such a manner that the formulation comprises one or more pharmaceutically acceptable excipients. Examples of some of the various classes or types of excipients that may be used in preparation of the dietary supplement include, but are not limited to, flavoring agents, flavor modifying agents (natural and synthetic), coloring agents, stabilizing agents, binders, disintegrants (both effervescent and non-effervescent), oral dispersing agents, glidants, preservatives, pH modifiers, stabilizers, diluents, compaction agents, lubricants, fillers, binders, taste-masking agents, flavor enhancing agents and other well-accepted types of excipients that are safe and effective for human use and consumption and combinations thereof. Because it is well understood that the number and type of specific excipients is too exhaustive and numerous to be listed here, it is to be understood that the inventors of the present invention have contemplated that the dietary supplement of the present invention may comprise any suitable combination of one or more pharmaceutically acceptable excipients, for instance, for preparation and manufacturing of the dietary supplement. Such representative excipients that may be used for preparation of the dietary supplement (for instance, for preparation of a suitable dosage form for administration of a dietary supplement) may include, but are not limited to, one or more of the pharmaceutically acceptable excipients disclosed in the monographs of the “Handbook of Pharmaceutical Excipients” (eight edition; edited by Sheskey, Cook, Walter and Colin), which is herein incorporated by reference.

It is further contemplated that, where appropriate and warranted, that one or more of the aforementioned ingredients is in the tautomeric, geometric or stereoisomeric equivalent form without departing from the spirit and scope of the present invention. The invention encompasses R- and S-enantiomers, cis- and trans-isomers, E- and Z-geometric isomers, diastereomers, d- and l-isomers, and racemic mixtures and combinations of mixtures thereof.

Additionally, one or more of the ingredients included in this combination may be in the form of a free base or in a pharmaceutically acceptable salt form prepared from either organic or inorganic acids.

### Dosage Forms

The dietary supplement that is the present invention may be produced and manufactured in one of a number of dosage forms, including but not limited to, the following oral dosage forms, or a combination thereof, including;
1. Tablets—wet granulations, dry granulations, hot melt extrusion, orally dispersible tablets, chewable tablets, disintegrating tablets, buccal tablets, and/or sublingual tablets;
2. Caplets—orally dispersible caplets, chewable caplets, disintegrating caplets, buccal caplets, and/or sublingual caplets;
3. Capsules—soft and hard gelatin;
4. Powders—orally dispersible powders, dissolving powders, melting powders, and/or effervescent powders);
5. Lozenges;
6. Sachets;
7. Troches;
8. Pellets;
9. Sprinkles;
10. Reconstitutable (powder) liquids;
11. Reconstitutable (powder) shakes;
12. Reconstitutable (powder) slurries;
13. Liquids—syrups, suspensions, elixirs, dispersions, polistirexes, emulsions, and/or solutions; and/or
14. Topical gels, ointments, and/or creams.

It is well understood in the art that different dosage forms require various dosing adjustments, regimens and routes of administration.

### Specified Ranges

Dosing for the provided ingredients is within the ranges below per pound weight:
Dosage ranges per pound are as follows:
Glucosamine—3 mg to 125 mg
Chondroitin—3 mg to 100 mg
Strontium Carbonate—2 mg to 7 mg
Vitamin A Palmitate—50 IU to 2000 IU
Vitamin D3—20 IU to 2000 IU
Vitamin K2 Mk7—0.2 mcg to 40 mcg

### Preferred Formulation

With regard to the present invention, dosing is achieved by measuring individualized dosages per one pound of body weight for mammalian consumption. Dosages per pound are as follows:
Glucosamine—9 mg
Chondroitin—7.2 mg
Strontium Carbonate—3.6 mg
Vitamin A Palmitate—260 i.u.
Vitamin D3—48 i.u.
Vitamin K2 Mk7—0.48 mcg

The aforementioned description of the preferred embodiments of the formulation of the present invention are presented for purposes of illustration and description and to impart to those having skill in the art a description necessary to make and practice the present invention. They are not intended to be exhaustive or to limit the present invention to the precise form and formulation disclosed. The exemplary formulation is chosen and described in order to best explain the principles of the present invention and its practical application thereby enabling those skilled in the art to best utilize the present invention exhibiting the disclosed micronutrients in combination.

I claim:

1. A chondroprotective and chondro-regenerative formulation consisting of the following ranges per pound body weight:
3-125 mg of glucosamine;
3-100 mg of chondroitin;
2-7 mg of strontium;
50-2000 IU of Vitamin A;
20-2000 IU of Vitamin D; and
0.2-40 mcg of Vitamin K.

2. The formulation of claim 1, wherein said ingredients are in a pure form or synthetic form.

3. The formulation of claim 1, wherein said formulation consists of the following per pound weight:
a. Glucosamine—9 mg
b. Chondroitin—7.2 mg
c. Strontium Carbonate—3.6 mg
d. Vitamin A Palmitate—260 IU
e. Vitamin D3—48 IU
f. Vitamin K2 Mk7—0.48 mcg

4. The formulation of claim 3 wherein:
a. glucosamine is glucosamine sulfate, glucosamine hydrochloride or N-Acetyl Glucosamine, D-glucosamine, D-glucosamine-sulfate, glucosamine potassium sulfate, glucosamine sulfate-potassium chloride, glucosamine-6-phosphate or a combination thereof.
b. chondroitin is chondroitin sulfate, calcium chondroitin sulfate, chondroitin polysulfate, Chondroitin Sulfate A, B or C, or a combination thereof.
c. strontium is strontium carbonate, strontium chloride, or combinations thereof.
d. Vitamin A is palmitate, beta carotene, retinyl, retinol, retinal and retinoic acid or combinations thereof.
e. Vitamin D is ergocalciferol (D₂), cholecalciferol (D₃), calcitriol or combinations thereof.
f. Vitamin K is a 2-methyl-1,4-naphthoquinone (3-) derivatives, Vitamin K2 (menaquinone), Vitamin K2 Mk4, Vitamin K2 Mk7, Vitamin K2 Mk11, Vitamin K3, Vitamin K4, Vitamin K5 or combinations thereof.

5. The formulation of claim 3, wherein the ingredients are in a pure form of synthetic form.

6. The formulation of claim 3, wherein said formulation further consists of pharmaceutically acceptable carrier, an excipient or a combination thereof.

7. The formulation of claim 3, wherein said formulation further consists of a flavoring agent, a coloring agent, a stabilizing agent, a binder, or a disintegrant.

8. The formulation of claim 3, wherein said formulation is administered orally, intramuscularly, intradermally, intravenously, nasally, subcutaneously or intraperitoneally concurrently or subsequently

9. A chondroprotective and chondro-regenerative formulation consisting essentially of the following per pound body weight:
3-125 mg of glucosamine;
3-100 mg of chondroitin;
2-7 mg of strontium;
50-2000 IU of Vitamin A;
20-2000 IU of Vitamin D; and
0.2-40 mcg of Vitamin K.

10. The formulation of claim 9, wherein said ingredients are in a pure form or synthetic form.

11. The formulation of claim 9, wherein said formulation consists essentially of the following per pound body weight:
a. Glucosamine—9 mg
b. Chondroitin—7.2 mg
c. Strontium Carbonate—3.6 mg
d. Vitamin A Palmitate—260 IU
e. Vitamin D3—48 IU
f. Vitamin K2 Mk7—0.48 mcg

12. The formulation of claim 9, wherein the ingredients are in a pure form of synthetic form.

13. The formulation of claim 9, wherein said formulation further consists of pharmaceutically acceptable carrier, an excipient or a combination thereof.

14. The formulation of claim 9, wherein said formulation further consists of: a flavoring agent, a coloring agent, a stabilizing agent, a binder, or a disintegrant.

15. The formulation of claim 9, wherein said formulation is administered orally, intramuscularly, intradermally, intravenously, nasally, subcutaneously or intraperitoneally concurrently or subsequently.

16. A method for supplying a dietary supplement and nutritional adjuvant in a specific formulation to a mammal to support the growth, maintenance and repair of bone and cartilage by supplying to a mammal:
chondroprotective, structure modifying osteoarthritis and symptomatic slow acting drugs in osteoarthritis entities in the form of glucosamine, chondroitin, in combination with strontium, Vitamin A, Vitamin D and Vitamin K, to support and maintain bone, synovial fluid, soft tissues and connective tissue and cartilage;
osteoclast suppressing agents in the form of strontium, in combination with glucosamine, chondroitin, Vitamin A, Vitamin D and Vitamin K, to improve cartilage metabolism, stimulate cartilage matrix formation, and promote bone growth and increase bone density;
unsaturated nutritional organic compounds that includes retinol, retinal, retinoic acid, and several provitamin A carotenoids in the form of the group constituting Vitamin A, in combination with glucosamine, chondroitin, strontium, Vitamin D and Vitamin K, to facilitate calcium absorption and metabolism and combine with Vitamin D, Vitamin K and a thyroid molecule, through heterodimerization, to prompt osteoblastic generating of bone preferentially over adipocyte formation;
Vitamin K, in combination with glucosamine, chondroitin, strontium, Vitamin D and Vitamin A, to promote bone formation and stem cell differentiation of stem cells into osteoblasts, through the combination with Vitamin D and Vitamin A, and through the upregulation of bone marker genes and functioning as an essential co-factor to bone proteins involved in calcium uptake, bone matrix mineralization and calcium ion homeostasis and inhibition of bone resorption, demineralization and soft tissue and cartilage support;
Vitamin D, in combination with glucosamine, chondroitin, strontium, Vitamin K and Vitamin A, to aid in the absorption and conservation of calcium, bone formation and muscle control and to work in combination with Vitamin K, Vitamin A and thyroid molecule to prompt osteoblastic generating of bone preferentially over adipocyte formation and to promote cell growth, stem cell regeneration, and retard inflammation while supporting proper neuromuscular and immune system functioning.

17. A method for supplying a dietary supplement and nutritional adjuvant in a specific formulation to a mammal to support the growth, maintenance and repair of bone and cartilage by supplying to a mammal in proportional amounts glucosamine, chondroitin, strontium, Vitamin A, Vitamin D and Vitamin K in combined, proportional amounts to work synergistically to effect chondroprotective and chondro-regenerative properties for bone, cartilage, and joints by (1) targeting degenerative bone disorders by strengthening bone and increasing bone density through prevention and maintenance, (2) addressing the etiology of osteoporosis by building and rebuilding cartilage, (3) increasing joint lubricity, and (4) aiding in the efficient and expedited repair of bone due to continual stress, degeneration, autoimmune disorder, infection or traumatic injury.

18. The method of claim 17, wherein said supplement delays bone resorption, potentiates osteoclast breakdown, and postpones progressive joint space narrowing through (1) stimulation of chondrocyte synthesis of collagen and proteoglycans, (2) enhanced synoviocyte production of hyaluronan, (3) inhibition of cartilage degradation, and (4) prevention of fibrin formation in the vasculature.

19. The method of claim 17, wherein said supplement is directed toward remediating the effects of osteoarthritis, osteoporosis, inflammatory joint disease, ankylosing spondylitis, various autoimmune disorders (e.g. rheumatoid arthritis, lupus, and Sjogrens syndrome), promoting preventative states of negative bone loss and/or enhanced bone deposition, and enhancing aiding in the promotion of the body's natural state of healing and rehabilitation of bone from mechanical wear, degeneration, strains, fractures, illness and bone breakage

20. The method of claim 17, where glucosamine, chondroitin, strontium, Vitamin A, Vitamin D and Vitamin K are supplied in the following forms and in the following amounts per pound weight:
Glucosamine—9 mg
Chondroitin—7.2 mg
Strontium Carbonate—3.6 mg
Vitamin A Palmitate—260 IU
Vitamin D3—48 IU
Vitamin K2 Mk7—0.48 mcg
